(19) United States
(12) Patent Application Publication
(10) Pub. No.: US 2009/0036386 A1
(43) Pub. Date: Feb. 5, 2009

(54) Heterobifunctional compounds for selectin inhibition

(75) Inventors: John L. Magnani, Gaithersburg, MD (US); Arun K. Sarkar, North Potomac, MD (US); John T. Patton, JR., Gaithersburg, MD (US)

Correspondence Address: SEED INTELLECTUAL PROPERTY LAW GROUP PLLC, 701 FIFTH AVE, SUITE 5400, SEATTLE, WA, 98104, US

(73) Assignee: GlycoMimetics, Inc, Gaithersburg, MD (US)

(21) Appl. No.: 11/920,499

(22) PCT Filed: May 24, 2006
(86) PCT No.: PCT/US2006/020249

§ 371 Date: Nov. 16, 2007

Related U.S. Application Data
(60) Provisional application No. 60/684,535, filed on May 25, 2005.

(51) Int. Cl. A61K 31/7042; C07H 17/04

(52) U.S. Cl. 514/27; 536/17.3

(57) ABSTRACT

Compounds and methods are provided for modulating in vitro and in vivo processes mediated by selectin binding. More specifically, selectin modulators and their use are described, wherein the selectin modulators that modulate (e.g., inhibit or enhance) a selectin-mediated function comprise glycomimetics linked to a compound, for example a member of a class of compounds termed BASAs (Benzyl Amino Sulfonic Acids) or a member of a class of compounds termed BACAs (Benzyl Amino Carboxylic Acids).

### BACKGROUND OF THE INVENTION

1. Field of the Invention

The present invention relates generally to compounds, compositions and methods for modulating processes mediated by selectin binding, and more particularly to selectin modulators and their use, wherein the selectin modulators that modulate a selectin-mediated function comprise particular glycomimetics linked to itself or another glycomimetic, to a member of a class of compounds termed BASAs (Benzyl Amino Sulfonic Acids), to a member of a class of compounds termed BACAs (Benzyl Amino Carboxylic Acids), or to orotic acid.

2. Description of the Related Art

When a tissue is infected or damaged, the inflammatory process directs leukocytes and other immune system components to the site of infection or injury. Within this process, leukocytes play an important role in the engulfment and digestion of microorganisms. Thus, the recruitment of leukocytes to infected or damaged tissue is critical for mounting an effective immune defense.

Selectins are a group of structurally similar cell surface receptors that are important for mediating leukocyte binding to endothelial cells. These proteins are type 1 membrane proteins and are composed of an amino terminal lectin domain, an epidermal growth factor (EGF)-like domain, a variable number of complement receptor related repeats, a hydrophobic domain spanning region and a cytoplasmic domain. The binding interactions appear to be mediated by contact of the lectin domain of the selectins and various carbohydrate ligands.

There are three known selectins: E-selectin, P-selectin and L-selectin. E-selectin is found on the surface of activated endothelial cells, which line the interior wall of capillaries. E-selectin binds to the carbohydrate sialyl-Lewis^{x} (SLeₓ), which is presented as a glycoprotein or glycolipid on the surface of certain leukocytes (monocytes and neutrophils) and helps these cells adhere to capillary walls in areas where surrounding tissue is infected or damaged; and E-selectin also binds to sialyl-Lewis^{a}(SLe^{a}), which is expressed on many tumor cells. P-selectin is expressed on inflamed endothelium and platelets, and also recognizes SLe^{x} and SLe^{a}, but also contains a second site that interacts with sulfated tyrosine. The expression of E-selectin and P-selectin is generally increased when the tissue adjacent to a capillary is infected or damaged. L-selectin is expressed on leukocytes. Selectin-mediated intercellular adhesion is an example of a selectin-mediated function.

Modulators of selectin-mediated function include the PSGL-1 protein (and smaller peptide fragments), fucoidan, glycyrrhizin (and derivatives), anti-selectin antibodies, sulfated lactose derivatives, and heparin. All have shown to be unsuitable for drug development due to insufficient activity, toxicity, lack of specificity, poor ADME characteristics and/or availability of material.

Although selectin-mediated cell adhesion is required for fighting infection and destroying foreign material, there are situations in which such cell adhesion is undesirable or excessive, resulting in tissue damage instead of repair. For example, many pathologies (such as autoimmune and inflammatory diseases, shock and reperfusion injuries) involve abnormal adhesion of white blood cells. Such abnormal cell adhesion may also play a role in transplant and graft rejection. In addition, some circulating cancer cells appear to take advantage of the inflammatory mechanism to bind to activated endothelium. In such circumstances, modulation of selectin-mediated intercellular adhesion may be desirable.

Accordingly, there is a need in the art for identifying inhibitors of selectin-mediated function, e.g., of selectin-dependent cell adhesion, and for the development of methods employing such compounds to inhibit conditions associated with excessive selectin activity. The present invention fulfills these needs and further provides other related advantages.

### BRIEF SUMMARY OF THE INVENTION

Briefly stated, this invention provides compounds, compositions and methods for modulating selectin-mediated processes. In the present invention, the compounds that modulate (e.g., inhibit or enhance) a selectin-mediated function comprise a particular glycomimetic linked to a BASA, to a BACA, to itself or another glycomimetic disclosed herein, or to orotic acid. Such compounds may be combined with a pharmaceutically acceptable carrier or diluent to form a pharmaceutical composition. The compounds or compositions may be used in a method to modulate (e.g., inhibit or enhance) a selectin-mediated function, such as inhibiting a selectin-mediated intercellular adhesion.

In one aspect of the present invention, compounds are provided having the formula:

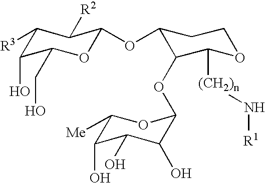

wherein:
n=0-20

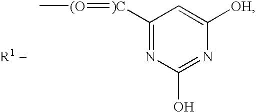

a benzyl amino sulfonic acid, a benzyl amino carboxylic acid, or a second compound or salt thereof having the above formula to form a dimer;

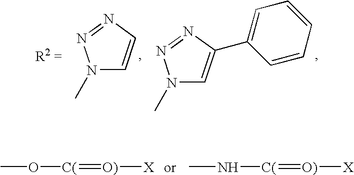

where X is

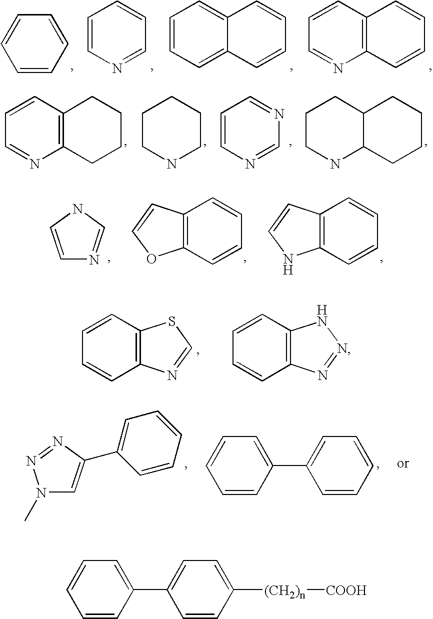

where n=0-10, and any of the above ring compounds may be substituted with one to three of Cl, F, C₁-C₈ alkanyl or OY where Y is H or C₁-C₈ alkanyl;

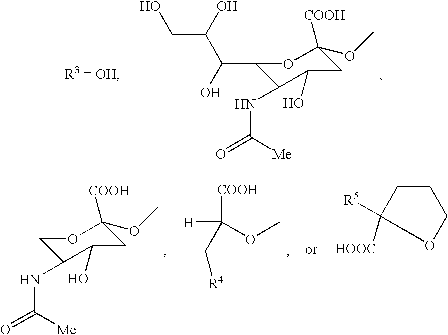

where R⁴ is cyclohexane, t-butane, adamantane, benzene, triazole, or triazole substituted with one to three of Cl, F, C₁-C₈ alkanyl or OY where Y is H or C₁-C₈ alkanyl, and where R⁵ is

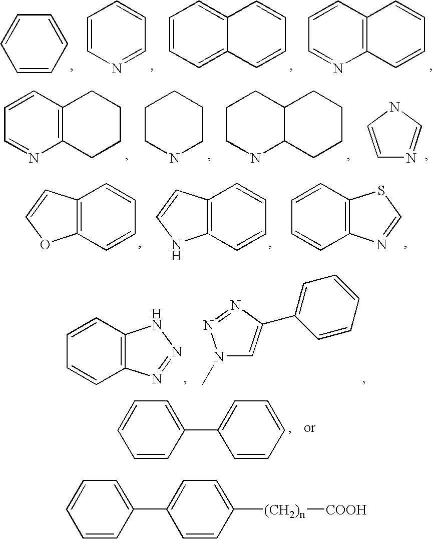

where n=0-10, and any one of the above ring compounds may be substituted with one to three of Cl, F, C₁-C₈ alkanyl or OY where Y is H or C₁-C₈ alkanyl; and
with the proviso that where R¹ is a benzyl amino sulfonic acid and R² or X of R² is aromatic, then R⁴ of R³ is not cyclohexane.

A compound of the present disclosure includes physiologically acceptable salts thereof. A compound of the present disclosure in combination with a pharmaceutically acceptable carrier or diluent provides a composition of the present invention. In the chemical formulae herein, a line extending from an atom depicted or from a carbon implied by the intersection of the two other lines, represents the point of attachment (and does not represent a methyl group). A methyl group is represented by “Me” or “CH₃”.

In an embodiment of the present invention, R¹ is a benzyl amino sulfonic acid.

In an embodiment, R¹ is a benzyl amino carboxylic acid.

In an embodiment, R³ is

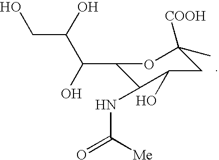

In an embodiment, R³ is

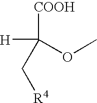

where R⁴ is defined as for the general formula above.

In an embodiment, R⁴ is cyclohexane or benzene.

In an embodiment, R¹ is

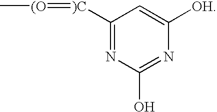

In an embodiment, R¹ is a second compound or salt thereof from the general formula above to form a dimer.

In an embodiment, X of R² is

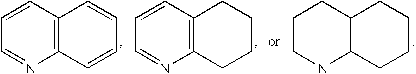

In an embodiment, R³ is OH.

In an embodiment, R³ is

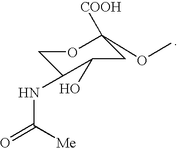

In an embodiment, X of R² is

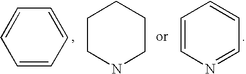

In an embodiment, X of R² is

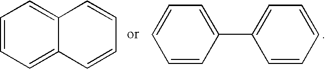

In an embodiment, R¹ is linked to the compound or salt thereof by a polyethylene glycol.

In an embodiment, a compound comprises a compound according to the present disclosure, further comprising a diagnostic or therapeutic agent. Such a compound may be combined with a pharmaceutically acceptable carrier or diluent to form one embodiment of a composition of the present invention.

In another aspect of the present invention, methods are provided for using a compound or composition of the present disclosure to modulate a selectin-mediated function. Such a compound or composition can be used, for example, to inhibit or enhance a selectin-mediated function, such as selectin-mediated intercellular interactions. A compound or composition can be used in a method to contact a cell expressing a selectin in an amount effective to modulate the selectin's function. A compound or composition can be used in a method to administer to a patient, who is in need of having inhibited the development of a condition associated with an excessive selectin-mediated function (such as an excessive selectin-mediated intercellular adhesion), in an amount effective to inhibit the development of such a condition. Examples of such conditions include inflammatory diseases, autoimmune diseases, infection, cancer, shock, thrombosis, wounds, burns, reperfusion injury, platelet-mediated diseases, leukocyte-mediated lung injury, spinal cord damage, digestive tract mucous membrane disorders, osteoporosis, arthritis, asthma and allergic reactions. A compound or composition can be used in a method to administer to a patient who is the recipient of a transplanted tissue in an amount effective to inhibit rejection of the transplanted tissue. A compound or composition can be used in a method in an amount effective to target an agent (e.g., a diagnostic or therapeutic agent) to a selectin-expressing cell by contacting such a cell with the agent linked to the compound or composition. A compound or composition can be used in the manufacture of a medicament, for example for any of the uses recited above.

These and other aspects of the present invention will become apparent upon reference to the following detailed description and attached drawings. All references disclosed herein are hereby incorporated by reference in their entirety as if each was incorporated individually.

### BRIEF DESCRIPTION OF THE DRAWINGS

FIGS. 1A and 1B are diagrams illustrating the syntheses of BASAs.

FIG. 2 is a diagram illustrating the synthesis of a BACA.

FIGS. 3A, 3B and 3C are diagrams illustrating the syntheses of PEGylated BASAs (XXXII and XXXIII) and PEGylated BACA (XXXVI).

FIGS. 4A-4E are diagrams illustrating the syntheses of selected compounds of the present disclosure and precursors thereto.

### DETAILED DESCRIPTION OF THE INVENTION

As noted above, the present invention provides selectin modulators, compositions thereof and methods for modulating selectin-mediated functions. Such modulators may be used in vitro or in vivo, to modulate (e.g., inhibit or enhance) selectin-mediated functions in a variety of contexts, discussed in further detail below. Examples of selectin-mediated functions include intercellular adhesion and the formation of new capillaries during angiogenesis.

### Selectin Modulators

The term “selectin modulator,” as used herein, refers to a molecule(s) that modulates (e.g., inhibits or enhances) a selectin-mediated function, such as selectin-mediated intercellular interactions. A selectin modulator may consist entirely of a glycomimetic compound of the present disclosure or glycomimetic compounds joined together, or may consist of such a glycomimetic linked to orotic acid, a BASA (Benzyl Amino Sulfonic Acid) or a BACA (Benzyl Amino Carboxylic Acid), or may comprise one or more additional molecular components to any of the above.

A selectin modulator of the present invention which does not possess a BASA or a BACA is preferably used to inhibit an E-selectin-mediated function. With the addition of a BASA or BACA to a glycomimetic of the present invention, the selectin modulator has increased ability to modulate P- or L-selectin-mediated functions (or both P- and L-selected-mediated functions) as well.

A selectin modulator of the present disclosure is a compound or physiologically acceptable salt thereof, having the formula:

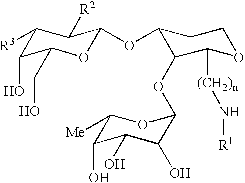

wherein:
n=0-20

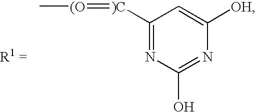

a benzyl amino sulfonic acid, a benzyl amino carboxylic acid, or a second compound or salt thereof having the above formula to form a dimer;

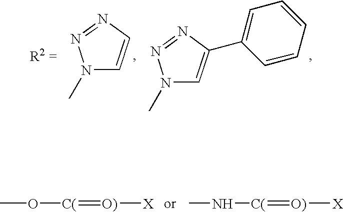

where X is

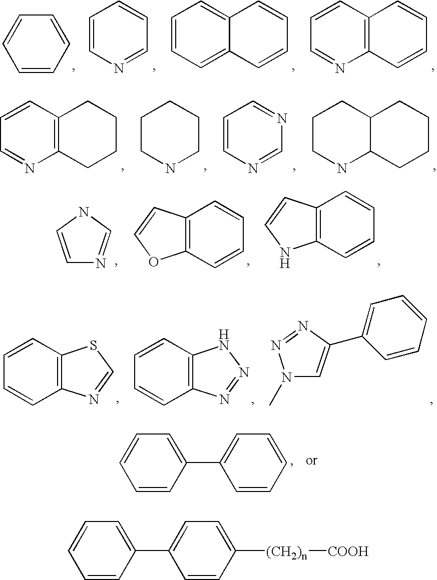

where n=0-10, and any of the above ring compounds may be substituted with one to three of Cl, F, C₁-C₈ alkanyl or OY where Y is H or C₁-C₈ alkanyl;

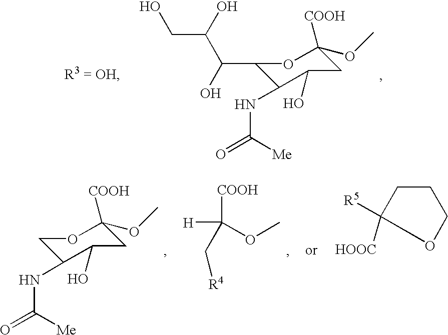

where R⁴ is cyclohexane, t-butane, adamantane, benzene, triazole, or triazole substituted with one to three of Cl, F, C₁-C₈ alkanyl or OY where Y is H or C₁-C₈ alkanyl, and where R⁵ is

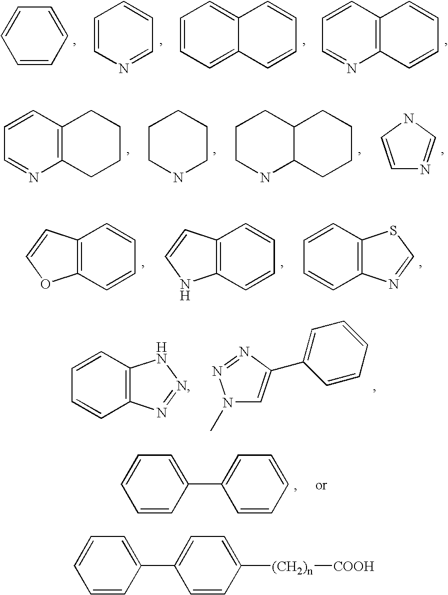

where n=0-10, and any one of the above ring compounds may be substituted with one to three of Cl, F, C₁-C₈ alkanyl or OY where Y is H or C₁-C₈ alkanyl; and
with the proviso that where R¹ is a benzyl amino sulfonic acid and R² or X of R² is aromatic, then R⁴ of R³ is not cyclohexane.

As used herein, a “C₁-C₈ alkanyl” refers to an alkane substituent with one to eight carbon atoms and may be straight chain or branched. Examples are methyl, ethyl, propyl, isopropyl, butyl and t-butyl. A “C₁-C₈ alkenyl” refers to an alkene substituent with one to eight carbon atoms, at least one carbon-carbon double bond, and may be straight chain or branched. Examples are similar to “C₁-C₈ alkanyl” examples except possessing at least one carbon-carbon double bond. A “C₁-C₁₄ aryl” refers to an aromatic (including heteroaromatic) substituent with one to fourteen carbon atoms in one or multiple rings which may be separated by a bond or fused. Examples are phenyl, naphthyl, pyridinyl, triazolo, furanyl, oxazolyl, thiophenyl, quinolinyl and diphenyl.

As used herein, polyethylene glycol (“PEG”) refers to multiple units of ethylene glycol, as well as those with one or more substituents (e.g., dicarboxylated PEG). PEGs with and without substituents are well known to those in the art. Within the present disclosure, PEG can serve as a substituent on a selectin modulator, or as a linker to attach other groups or compounds to a selectin modulator, or a selectin modulator may possess more than one PEG.

Where a second selectin modulator is linked at R¹ to a first selectin modulator, a dimer of selectin modulators (i.e., a divalent molecule) is formed. A variety of linkers, including those described herein, may be used to join the two selectin modulators. For example, PEG may be used as the linker to prepare a dimer. As used herein, a “dimer” can be a homodimer or a heterodimer. A homodimer refers to a dimer where the two selectin modulators joined together are identical (independent of the substituents for the linking to one another). A heterodimer refers to a dimer where the two selectin modulators (independent of the linkage substituents) are not identical.

Alternatively, the following orotic acid may be added at R¹:

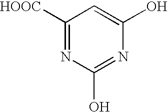

A variety of linkers, including those described herein, may be used to add the orotic acid. In an embodiment, the orotic acid is joined via its carboxylic acid group. A linker need not be used to couple the orotic acid at R¹. For example, the carboxylic acid group of the orotic acid may be coupled directly (via an appropriate catalyst) or converted to an acid halide or an activated ester, and then reacted with the —NH₂ at R¹.

A selectin modulator of the present disclosure may possess, at R³ of the above formula, sialic acid or a sialic acid mimic as set forth above. For example, the hexose ring of sialic acid may be replaced with cyclohexane. The presence of sialic acid in the selectin modulator enhances P-selectin binding. Where only E-selectin binding (and not both E- and P-selecting binding) is desired, a sialic acid mimic replaces sialic acid in the selectin modulator.

Alternative to (or in combination with) the replacement of a sialic acid mimic with sialic acid, P-selectin binding may be enhanced by the addition of a BASA or a BACA. As disclosed above, the selectin modulator compounds of the present disclosure may possess at R¹ a BASA or a BACA. The addition of a BASA or BACA to a selectin modulator compound of the present disclosure that lacks sialic acid, may convert the selectin modulator from a compound that is selective for binding to E-selectin to one that binds either E- or P-selectin or both (or enhances the binding to E- or P-selectin or both). BASA or BACA includes a portion or an analogue of a BASA or BACA or portion of either analogue, provided that the compound retains the ability to modulate a selectin-mediated function. PEG may be added to a selectin modulator with or without a BASA (or BACA). PEG may also be used to link a BASA or BACA to a selectin modulator.

Within the present disclosure, BASAs are low molecular weight sulfated compounds which have the ability to interact with a selectin. The interaction modulates or assists in the modulation (e.g., inhibition or enhancement) of a selectin-mediated function (e.g., an intercellular interaction). They exist as either their protonated acid form, or as a sodium salt, although sodium may be replaced with potassium or any other pharmaceutically acceptable counterion. A representative BASA has the following structure:

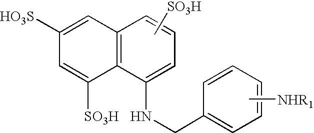

Portions of BASA that retain the ability to interact with a selectin (which interaction modulates or assists in the modulation of a selectin-mediated function as described herein) are also a BASA component of the selectin modulators of the present invention. Such portions generally comprise at least one aromatic ring present within the BASA structure. Within certain embodiments, a portion may comprise a single aromatic ring, multiple such rings or half of a symmetrical BASA molecule.

As noted above, analogues of BASA and portions thereof (both of which possess the biological characteristic set forth above) are also encompassed, e.g., by the BASA component of the selectin modulators, within the present invention. As used herein, an “analogue” is a compound that differs from BASA or a portion thereof because of one or more additions, deletions and/or substitutions of chemical moieties, such that the ability of the analogue to inhibit a selectin-mediated interaction is not diminished. For example, an analogue may contain S to P substitutions (e.g., a sulfate group replaced with a phosphate group). Other possible modifications include: (a) modifications to ring size (e.g., any ring may contain between 4 and 7 carbon atoms); (b) variations in the number of fused rings (e.g., a single ring may be replaced with a polycyclic moiety containing up to three fused rings, a polycyclic moiety may be replaced with a single unfused ring or the number of fused rings within a polycyclic moiety may be altered); (c) ring substitutions in which hydrogen atoms or other moieties covalently bonded to a carbon atom within an aromatic ring may be replaced with any of a variety of moieties, such as F, Cl, Br, I, OH, O-alkyl (C1-8), SH, NO₂, CN, NH₂, NH-alkyl (C1-8), N-(alkyl)₂, SO₃M (where M═H⁺, Na⁺, K⁺ or other pharmaceutically acceptable counterion), CO₂M, PO₄M₂, SO₂NH₂, alkyl (C1-8), aryl (C6-10), CO₂-alkyl (C1-8), —CF₂X (where X can be H, F, alkyl, aryl or acyl groups) and carbohydrates; and (d) modifications to linking moieties (i.e., moieties located between rings in the BASA molecule) in which groups such as alkyl, ester, amide, anhydride and carbamate groups may be substituted for one another.

Certain BASA portions and analogues contain one of the following generic structures:

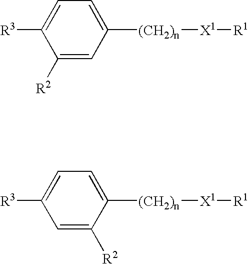

Within this structure, n may be 0 or 1, X¹ may be —PO₂M, —SO₂M or —CF₂— (where M is a pharmaceutically acceptable counterion such as hydrogen, sodium or potassium), R¹ may be —OH, —F or —CO₂R⁴ (where R⁴ may be —H or —(CH₂)ₘ—CH₃ and m is a number ranging from 0 to 3, R² may be —H, —PO₃M₂, —SO₃M₂, —CH₂—PO₃M₂, —CH₂—SO₃M₂, —CF₃ or —(CH₂)ₘ—C(R⁶)H—R⁵ or R⁹—N(R¹⁰)—, R³ may be —H, —(CH₂)ₘ—C(R⁶)H—R⁵ or R⁹—N(R¹⁰)— (where R⁵ and R⁶ may be independently selected from —H, —CO₂—R⁷ and —NH—R⁸, R⁷ and R⁸ may be independently selected from hydrogen and moieties comprising one or more of an alkyl group, an aromatic moiety, an amino group or a carboxy group, and R⁹ and R¹⁰ may be independently selected from —H, —(CH₂)ₘ—CH₃; —CH₂—Ar, —CO—Ar, where m is a number ranging from 0 to 3 and Ar is an aromatic moiety (i.e., any moiety that comprises at least one substituted or unsubstituted aromatic ring, wherein the ring is directly bonded to the —CH₂— or —CO— group indicated above)).

Other portions and analogues of BASA comprise the generic structure:

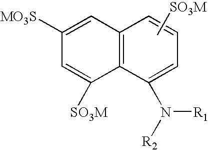

Within this structure, R₁ and R₂ may be independently selected from (i) hydrogen, (ii) moieties comprising one or more of an alkyl group, an aromatic moiety, an amino group or a carboxy group, and (iii) —CO—R₃ (where R₃ comprises an alkyl or aromatic moiety as described above) and M is a pharmaceutically acceptable counterion.

The individual compounds, or groups of compounds, derived from the various combinations of the structures and substituents described herein, are disclosed by the present application to the same extent as if each compound or group of compounds was set forth individually. Thus, selection of particular structures and/or particular substituents is within the scope of the present invention.

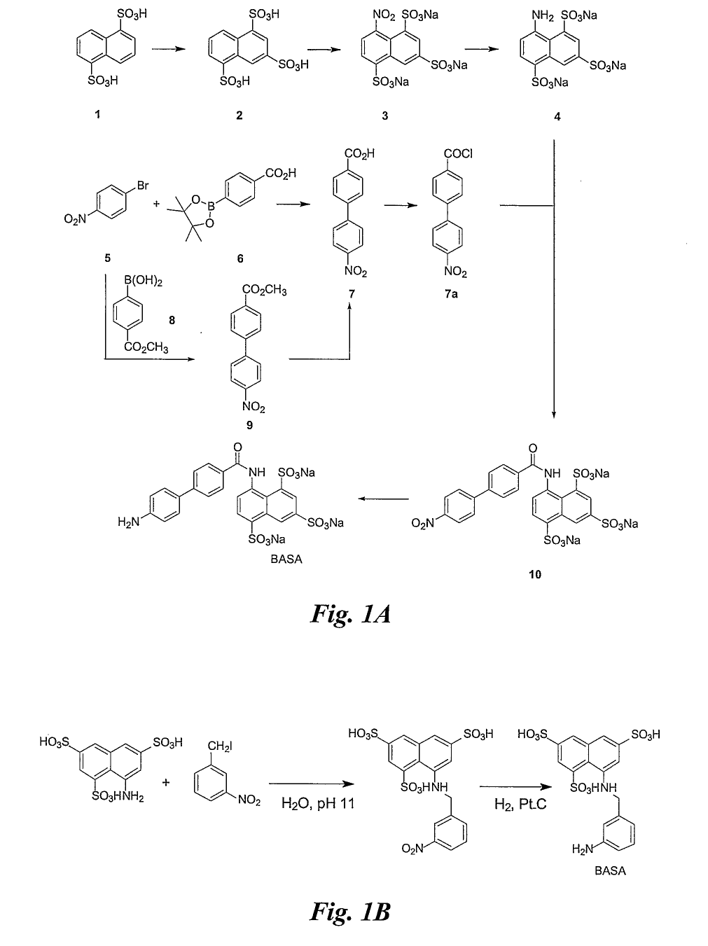

Representative BASA portions and analogues are included in the compounds shown in FIGS. 1A-1B. It will be apparent to those of ordinary skill in the art that modifications may be made to the compounds shown within these figures, without adversely affecting the ability to function as selectin modulators. Such modifications include deletions, additions and substitutions as described above.

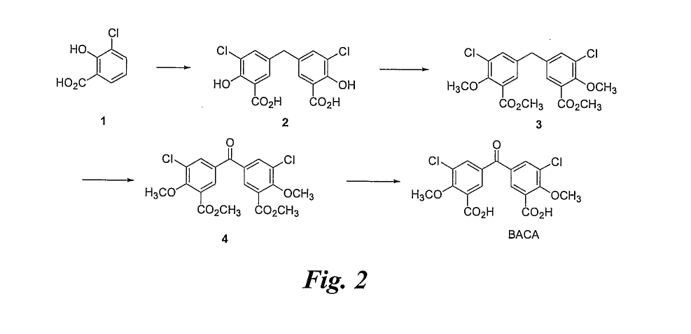

A BACA is similar to a BASA, except instead of sulfonic acid groups, the compound possesses carboxylic acid groups. A representative BACA is shown in FIG. 2. For example, the sulfonic acid groups of the above BASA compounds may be replaced with carboxylic acid groups. Thus, the above disclosure to BASAs is incorporated by reference into this description of BACAs.

Examples of BACAs include:

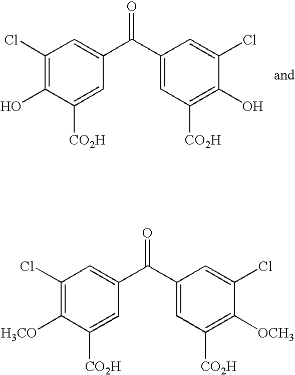

As described above, a BASA or BACA may be joined to a compound of the present invention at R¹ via a linker. Typically a linker is first attached to one of a glycomimetic or a BASA/BACA, which is then reacted with the other. The attachment of a BASA or BACA to a particular glycomimetic can be accomplished in a variety of ways to form a selectin modulator. A linker possessed by (or added to) a BASA or BACA or a glycomimetic may include a spacer group, such as —(CH₂)— or —(CH₂)— where n is generally about 1-20 (including any whole integer range therein). An example of a linker is —NH₂ on a glycomimetic, e.g., CH2—NH₂ when it includes a short spacer group. In an embodiment, —CH₂—NH₂ is attached to a glycomimetic at R¹ which may then be used to attach a BASA or BACA. The —NH₂ may be formed by reduction of —N₃ (azido group). A simple method of attachment of a glycomimetic possessing —NH₂ is to react with a BASA or BACA which possesses (or has been modified to possess) an electrophilic group that is susceptible to nucleophilic attack by the —NH₂ on the glycomimetic. Another simple attachment method is reductive amination of the BASA or BACA to a glycomimetic containing a reducing end (an anomeric hydroxyl/aldehyde). This is accomplished by simple reaction of the BASA or BACA to the reducing end and subsequent reduction (e.g., with NaCNBH₃ at pH 4.0) of the imine formed. The most general approach entails the simple attachment of an activated linker to the glycomimetic via an O, S or N heteroatom (or C atom) at the anomeric position. The methodology of such attachments has been extensively researched for carbohydrates and anomeric selectivity is easily accomplished by proper selection of methodology and/or protecting groups. Examples of potential glycosidic synthetic methods include Lewis acid catalyzed bond formation with halogen or peracetylated sugars (Koenigs Knorr), trichloroacetamidate bond formation, thioglycoside activation and coupling, glucal activation and coupling, n-pentenyl coupling, phosphonate ester homologation (Horner-Wadsworth-Emmons reaction), and many others. Alternatively, linkers could be attached to positions on the moieties other than the anomeric. The most accessible site for attachment is at a six hydroxyl (6-OH) position of a glycomimetic (a primary alcohol). The attachment of a linker at the 6-OH can be easily achieved by a variety of means. Examples include reaction of the oxy-anion (alcohol anion formed by deprotonation with base) with an appropriate electrophile such as an alkyl/acyl bromide, chloride or sulfonate ester, activation of the alcohol via reaction with a sulfonate ester chloride or POCl₃ and displacement with a subsequent nucleophile, oxidation of the alcohol to the aldehyde or carboxylic acid for coupling, or even use of the Mitsunobu reaction to introduce differing functionalities. Once attached the linker is then functionalized for reaction with a suitable nucleophile on the BASA or BACA (or vice versa). This is often accomplished by use of thiophosgene and amines to make thiourea-linked heterobifunctional ligands, diethyl squarate attachment (again with amines) and/or simple alkyl/acylation reactions. Additional methods that could be utilized include FMOC solid or solution phase synthetic techniques traditionally used for carbohydrate and peptide coupling and chemo-enzymatic synthesis techniques possibly utilizing glycosyl/fucosyl transferases and/or oligosaccharyltransferase (OST).

Embodiments of linkers include the following:

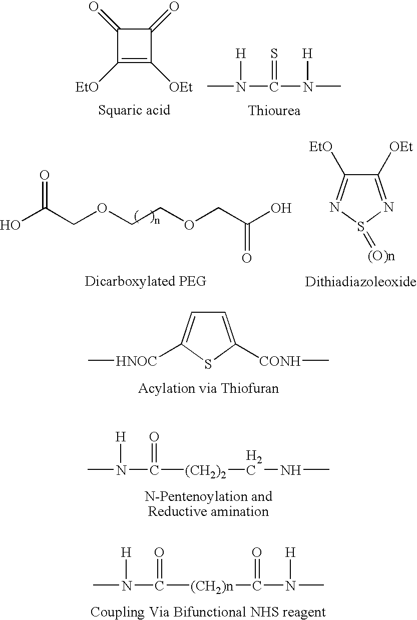

Other linkers will be familiar to those in the art.

A compound, or physiologically acceptable salt thereof, of the present invention has the formula:

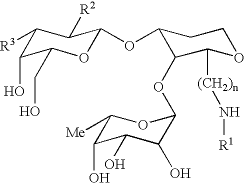

wherein R¹-R³ are defined as set forth above.

In an embodiment, R¹ is a benzyl amino sulfonic acid. In an embodiment, R¹ is a benzyl amino carboxylic acid. In an embodiment, R³ is

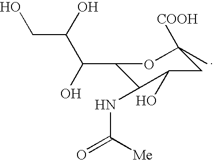

In an embodiment, R³ is

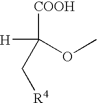

where R⁴ is defined as above. In an embodiment, R⁴ is cyclohexane or benzene. In an embodiment, R¹ is

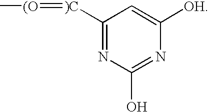

In an embodiment, R¹ is a second compound or salt thereof from the general formula above to form a dimer. In an embodiment, X of R² is

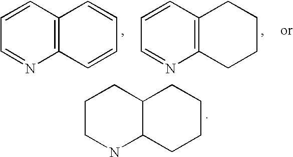

In an embodiment, R³ is OH. In an embodiment, R³ is

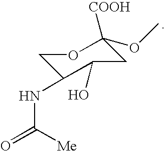

In an embodiment, X of R² is

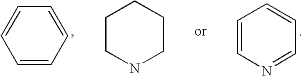

In an embodiment, X of R² is

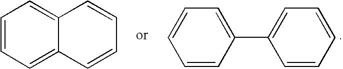

In an embodiment, R¹ is linked to the compound or salt thereof by a polyethylene glycol.

Although selectin modulators as described herein may sufficiently target a desired site in vivo, it may be beneficial for certain applications to include an additional targeting moiety to facilitate targeting to one or more specific tissues. As used herein, a “targeting moiety,” may be any substance (such as a compound or cell) that, when linked to a modulating agent enhances the transport of the modulator to a target tissue, thereby increasing the local concentration of the modulator. Targeting moieties include antibodies or fragments thereof, receptors, ligands and other molecules that bind to cells of, or in the vicinity of, the target tissue. Linkage is generally covalent and may be achieved by, for example, direct condensation or other reactions, or by way of bi- or multi-functional linkers.

For certain embodiments, it may be beneficial to also, or alternatively, link a drug to a selectin modulator. As used herein, the term “drug” refers to any bioactive agent intended for administration to a mammal to prevent or treat a disease or other undesirable condition. Drugs include hormones, growth factors, proteins, peptides and other compounds. Examples of potential drugs include antineoplastic agents (such as 5-fluorouracil and distamycin), integrin agonist/antagonists (such as cyclic-RGD peptide), cytokine agonist/antagonists, histamine agonist/antagonists (such as diphenhydramine and chlorpheniramine), antibiotics (such as aminoglycosides and cephalosporins) and redox active biological agents (such as glutathione and thioredoxin). In other embodiments, diagnostic or therapeutic radionuclides may be linked to a selectin modulator. In many embodiments, the agent may be linked directly or indirectly to a selectin modulator.

Modulators as described herein may be present within a pharmaceutical composition. A pharmaceutical composition comprises one or more modulators in combination with one or more pharmaceutically or physiologically acceptable carriers, diluents or excipients. Such compositions may comprise buffers (e.g., neutral buffered saline or phosphate buffered saline), carbohydrates (e.g., glucose, mannose, sucrose or dextrans), mannitol, proteins, polypeptides or amino acids such as glycine, antioxidants, chelating agents such as EDTA or glutathione, adjuvants (e.g., aluminum hydroxide) and/or preservatives. Within yet other embodiments, compositions of the present invention may be formulated as a lyophilizate. Compositions of the present invention may be formulated for any appropriate manner of administration, including for example, topical, oral, nasal, intravenous, intracranial, intraperitoneal, subcutaneous, or intramuscular administration.

A pharmaceutical composition may also, or alternatively, contain one or more active agents, such as drugs (e.g., those set forth above), which may be linked to a modulator or may be free within the composition.

The compositions described herein may be administered as part of a sustained release formulation (i.e., a formulation such as a capsule or sponge that effects a slow release of modulating agent following administration). Such formulations may generally be prepared using well known technology and administered by, for example, oral, rectal or subcutaneous implantation, or by implantation at the desired target site. Carriers for use within such formulations are biocompatible, and may also be biodegradable; preferably the formulation provides a relatively constant level of modulating agent release. The amount of modulating agent contained within a sustained release formulation depends upon the site of implantation, the rate and expected duration of release and the nature of the condition to be treated or prevented.

Selectin modulators are generally present within a pharmaceutical composition in a therapeutically effective amount. A therapeutically effective amount is an amount that results in a discernible patient benefit, such as increased healing of a condition associated with excess selectin-mediated function (e.g., intercellular adhesion), as described below.

In general, the modulating agents and compositions described herein may be used for enhancing or inhibiting a selectin-mediated function. Such enhancement or inhibition may be achieved in vitro and/or in vivo in a warm-blooded animal, preferably in a mammal such as a human, provided that a selectin is ultimately contacted with a modulator, in an amount and for a time sufficient to enhance or inhibit selectin-mediated function.

Within certain aspects, the present invention provides methods for inhibiting the development of a condition associated with a selectin-mediated function, such as intercellular adhesion. In general, such methods may be used to prevent, delay or treat such a condition. In other words, therapeutic methods provided herein may be used to treat a disease, or may be used to prevent or delay the onset of such a disease in a patient who is free of disease or who is afflicted with a disease that is not associated with a selectin-mediated function. For example, the therapeutic methods have uses that may include the arrest of cell growth, the killing of cells, the prevention of cells or cell growth, the delay of the onset of cells or cell growth, or the prolongation of survival of an organism.

A variety of conditions are associated with a selectin-mediated function. Such conditions include, for example, tissue transplant rejection, platelet-mediated diseases (e.g., atherosclerosis and clotting), hyperactive coronary circulation, acute leukocyte-mediated lung injury (e.g., adult respiratory distress syndrome (ARDS)), Crohn's disease, inflammatory diseases (e.g., inflammatory bowel disease), autoimmune diseases (MS, myasthenia gravis), infection, cancer (and metastasis), thrombosis, wounds (and wound-associated sepsis), burns, spinal cord damage, digestive tract mucous membrane disorders (gastritis, ulcers), osteoporosis, rheumatoid arthritis, osteoarthritis, asthma, allergy, psoriasis, septic shock, traumatic shock, stroke, nephritis, atopic dermatitis, frostbite injury, adult dyspnoea syndrome, ulcerative colitis, systemic lupus erythematosus, diabetes and reperfusion injury following ischaemic episodes. Selectin modulators may also be administered to a patient prior to heart surgery to enhance recovery. Other uses include pain management, prevention of restinosis associated with vascular stenting, and for undesirable angiogenesis, e.g., associated with cancer.

Selectin modulators of the present invention may be administered in a manner appropriate to the disease to be treated (or prevented). Appropriate dosages and a suitable duration and frequency of administration may be determined by such factors as the condition of the patient, the type and severity of the patient's disease and the method of administration. In general, an appropriate dosage and treatment regimen provides the modulating agent(s) in an amount sufficient to provide therapeutic and/or prophylactic benefit. Within particularly preferred embodiments of the invention, a selectin modulator may be administered at a dosage ranging from 0.001 to 1000 mg/kg body weight (more typically 0.01 to 1000 mg/kg), on a regimen of single or multiple daily doses. Appropriate dosages may generally be determined using experimental models and/or clinical trials. In general, the use of the minimum dosage that is sufficient to provide effective therapy is preferred. Patients may generally be monitored for therapeutic effectiveness using assays suitable for the condition being treated or prevented, which will be familiar to those of ordinary skill in the art.

Selectin modulators may also be used to target substances to cells that express a selectin. Such substances include therapeutic agents and diagnostic agents. Therapeutic agents may be a molecule, virus, viral component, cell, cell component or any other substance that can be demonstrated to modify the properties of a target cell so as to provide a benefit for treating or preventing a disorder or regulating the physiology of a patient. A therapeutic agent may also be a prodrug that generates an agent having a biological activity in vivo. Molecules that may be therapeutic agents may be, for example, polypeptides, amino acids, nucleic acids, polynucleotides, steroids, polysaccharides or inorganic compounds. Such molecules may function in any of a variety of ways, including as enzymes, enzyme inhibitors, hormones, receptors, antisense oligonucleotides, catalytic polynucleotides, anti-viral agents, anti-tumor agents, anti-bacterial agents, immunomodulating agents and cytotoxic agents (e.g., radionuclides such as iodine, bromine, lead, palladium or copper). Diagnostic agents include imaging agents such as metals and radioactive agents (e.g., gallium, technetium, indium, strontium, iodine, barium, bromine and phosphorus-containing compounds), contrast agents, dyes (e.g., fluorescent dyes and chromophores) and enzymes that catalyze a calorimetric or fluorometric reaction. In general, therapeutic and diagnostic agents may be attached to a selectin modulator using a variety of techniques such as those described above. For targeting purposes, a selectin modulator may be administered to a patient as described herein. Since selectins are expressed on endothelial cells involved in the formation of new capillaries during angiogenesis, a selectin modulator may be used to target a therapeutic agent for killing a tumor's vasculature. A selectin modulator may also be used for gene targeting.

Selectin modulators may also be used in vitro, e.g., within a variety of well known cell culture and cell separation methods. For example, modulators may be linked to the interior surface of a tissue culture plate or other cell culture support, for use in immobilizing selectin-expressing cells for screens, assays and growth in culture. Such linkage may be performed by any suitable technique, such as the methods described above, as well as other standard techniques. Modulators may also be used, for example, to facilitate cell identification and sorting in vitro, permitting the selection of cells expressing a selectin (or different selectin levels). Preferably, the modulator(s) for use in such methods are linked to a detectable marker. Suitable markers are well known in the art and include radionuclides, luminescent groups, fluorescent groups, enzymes, dyes, constant immunoglobulin domains and biotin. Within one preferred embodiment, a modulator linked to a fluorescent marker, such as fluorescein, is contacted with the cells, which are then analyzed by fluorescence activated cell sorting (FACS).

Modulating agents as described above are capable, for example, of inhibiting selectin-mediated cell adhesion. This ability may generally be evaluated using any of a variety of in vitro assays designed to measure the effect on adhesion between selectin-expressing cells (e.g., adhesion between leukocytes or tumor cells and platelets or endothelial cells). For example, such cells may be plated under standard conditions that, in the absence of modulator, permit cell adhesion. In general, a modulator is an inhibitor of selectin-mediated cell adhesion if contact of the test cells with the modulator results in a discernible inhibition of cell adhesion. For example, in the presence of modulators (e.g., micromolar levels), disruption of adhesion between leukocytes or tumor cells and platelets or endothelial cells may be determined visually within approximately several minutes, by observing the reduction of cells interacting with one another.

All compounds of the present invention or useful thereto, include physiologically acceptable salts thereof.

The following Examples are offered by way of illustration and not by way of limitation.

### EXAMPLES

### Example 1

### Synthesis of BASA (FIG. 1A)

Synthesis of compound 4: Nitration of commercially available 2 (1g) is according to the procedure described (for literature conditions see U.S. Pat. No. 4,534,905; Allison, F. et al. Helv. Chim. Acta 4:2139 (1952)).

The crude product 3 is dissolved in water (40 mL) and 10% Pd/C (0.3 g) added. The mixture is hydrogenated (˜45 psi) at room temperature for 48 h. The catalyst is filtered through Celite and the filter bed is washed with water. The filtrate is concentrated under vacuum to afford a pink solid. After removal of the catalyst, the filtrate is concentrated to 15 mL and an equal volume of ethanol is added. The precipitate is collected by filtration to give compound 4 with very little impurity.

Synthesis of compound 7a: A solution of 5 (5g) and 8 (4.45 g, 24.7 mmol), and K₂CO₃ (2 M in H₂O, 24.7 mL, 49.4 mmol) in 10:1 toluene/ethanol (70 mL) is treated with Pd(PPh₃)₄ (1.43 g, 1.24 mmol) and the mixture is refluxed for 20 h. After work up, recrystallization of the crude product in EtOH and chromatographic purification of the recrystallization filtrate affords compound 9 (2.9 g, 46%, >90% HPLC) and 2.2 g of recovered 5. The product is characterized by ¹H NMR.

A mixture of 9 (2.9 g, 11.3 mmol) and LiOH.H₂O (1.43 g, 34.1 mmol) in 1:1 THF/H₂O (250 mL) is stirred at RT for 21 h. The reaction affords 7 (2.58 g, 94%, >90% HPLC) after work up. The product is characterized by ¹H NMR.

DMF (20 μl) is added to a suspension of 7 (500 mg, 1.94 mmol), SOCl₂ (0.23 mL, 3.10 mmol) and toluene (3 mL) and then heated to 80° C. After 20 h the reaction is worked up to afford the acid chloride (640 mg). The product 7a is characterized by IR and ¹H NMR.

Synthesis of compound 10: To a solution of amine 4 (268 mg, 0.641 mmol) in H₂O (2 mL) and dioxane (18 mL) is added a solution of 7a (273 mg, 0.99 mmol) in dioxane (16 mL) dropwise over 30 min. The pH of the reaction mixture is adjusted to 8.5 with 0.25 M NaOH as the addition progresses. The reaction is stirred at room temperature for 2.5 h after the addition. Purification by column chromatography (methanol/toluene 1:1) followed by prep. TLC (methanol/toluene 1:1) affords 50 mg of compound 10, which is characterized by ¹H NMR and MS.

Hydrogenation of compound 10: A suspension of 10 (30 mg, 0.049 mmol) and 10% Pd on carbon (50 mg) in H₂O (20 mL) is hydrogenated (55 psi) at room temperature for 4 h to yield the BASA of FIG. 1A.

### Example 2

### Synthesis of BASA (FIG. 1B)

Synthesis of compound xxxxv: 3-nitro-benzyl iodide is added to an aqueous solution (pH 11) of commercially available, 8-aminonaphthalene-1,3,5-trisulfonic acid (xxxxxi) with stirring at room temperature. pH of the solution is adjusted to 1 and after evaporation of the solvent, the product xxxxiii is precipitated out from ethanol.

Platinum catalyzed hydrogenation of compound xxxxiii affords compound xxxxiv (the BASA of FIG. 1B) in 96% yield.

### Example 3

### Synthesis of BACA (FIG. 2)

A suspension of 1 (8.9 g), paraformaldehyde (8.9 g), and H₂SO₄ (125 mL) is heated to 90° C. for 14 h and affords crude 2 (7.8 g) after work up. The crude product is 77% pure by HPLC and characterized by ¹H NMR.

To a solution of 2 (1.0 g) in acetone (30 mL) is added K₂CO₃ (3.1 g) and dimethylsulfate (1.4 mL) and the reaction is heated to reflux for 24 h. The reaction is combined with the next batch for work up and purification.

To a solution of 2 (7.5 g) in acetone (225 mL) is added K₂CO₃ (23.2 g) and dimethylsulfate (10.8 mL) and the reaction is heated to reflux for 16 h. The reaction, combined with the previous batch, affords 3 (7.3 g, 74%) after work up and column chromatographic purification (ethyl acetate/heptane 1:9). The product is 80% pure by HPLC and characterized by ¹H NMR.

Chromic anhydride (6.94 g) is added to a suspension of 3 (7.16 g) in acetic anhydride (175 mL) at 3° C. and then stirred at room temperature for 15 h. The reaction affords 4 (5.89 g) after work up and column purification (100% dichloromethane). The product is 90% pure by HPLC and characterized by ¹H NMR.

To a suspension of 4 (5.89 g) in THF/H₂O (300 mL, 1:1) is added LiOH H₂O (1.74 g) at room temperature and the resulting mixture is stirred for 14 h. After an acid/base work up, the product is obtained as a white solid. The product is dried under high vacuum and characterized by nmr and mass spectroscopy.

### Example 4

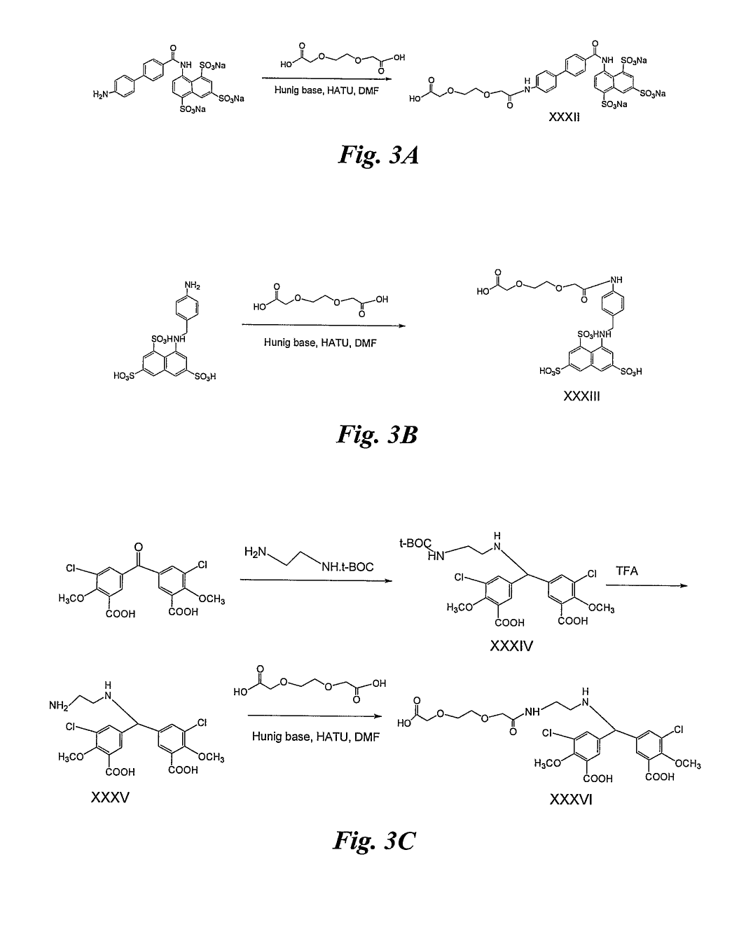

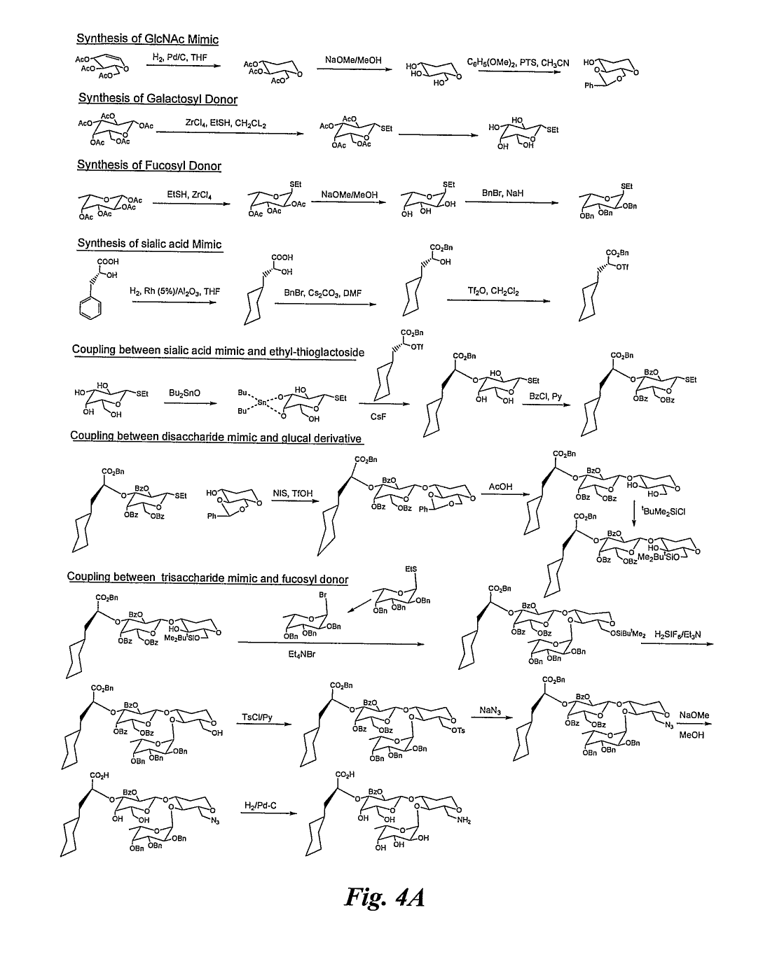

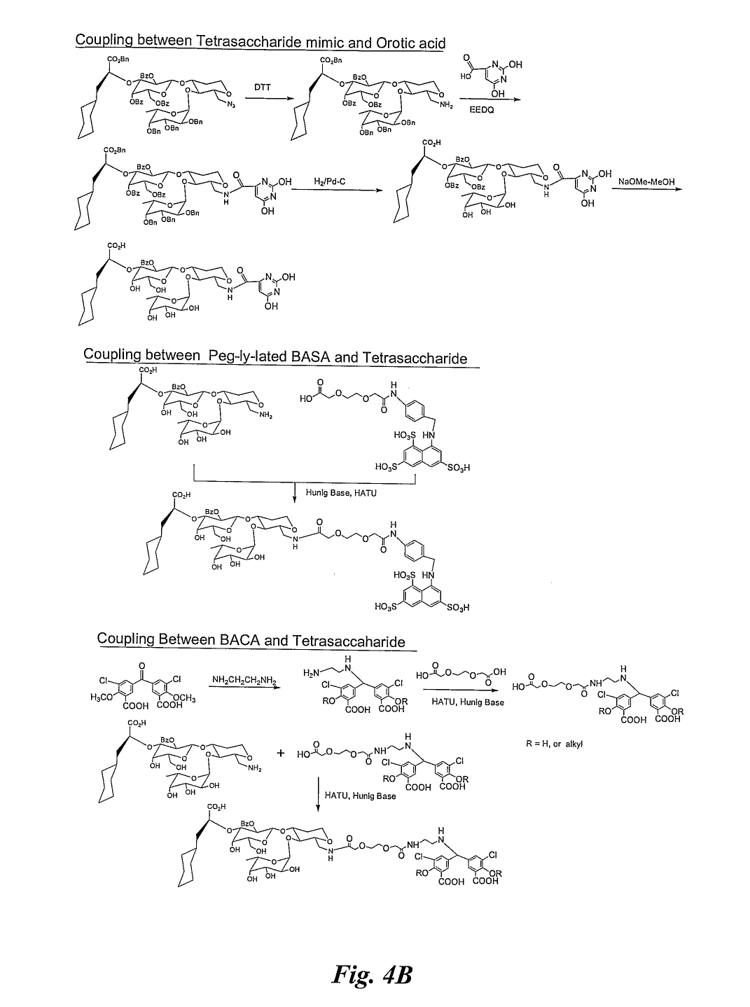

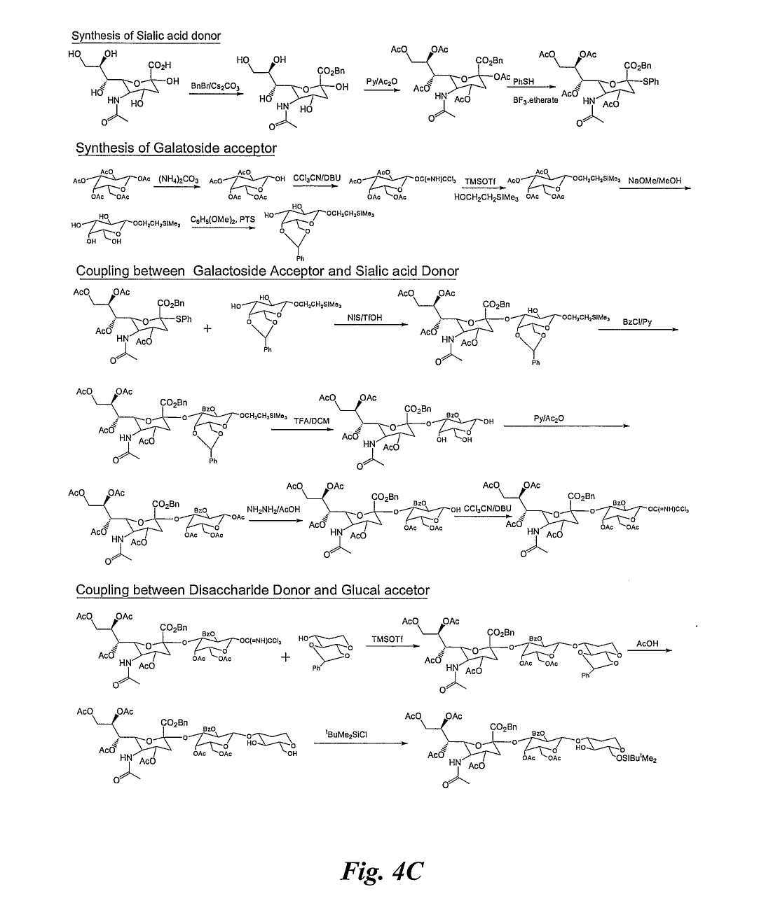

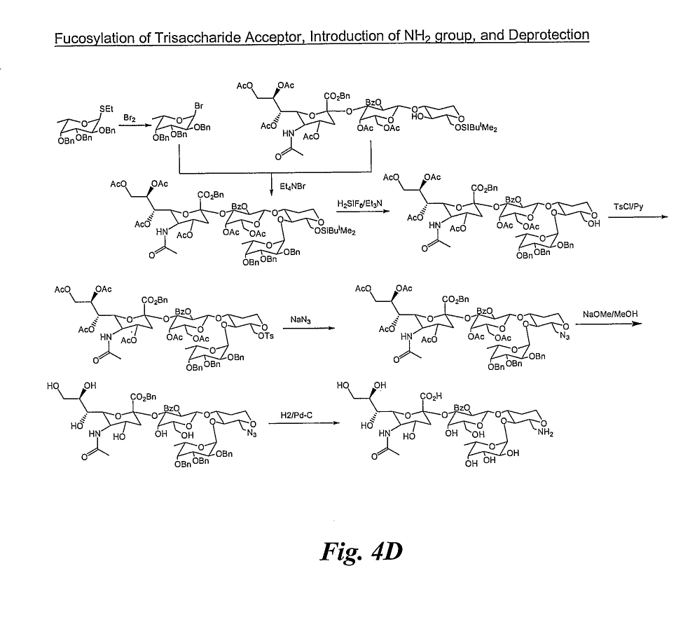

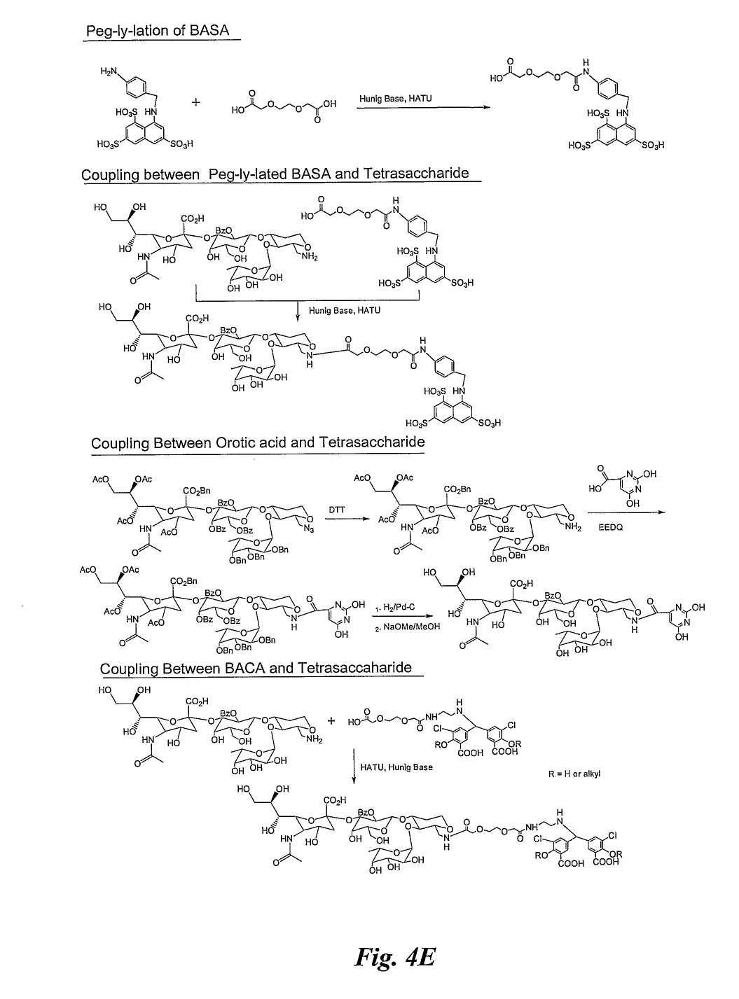

### Synthesis of PEGylated BASA (FIG. 3B)

To a solution of PEG (200 mg) in DMF (1 ml) is added Hunig base (g), and then HATU (g) is added after 5 min. The solution is stirred at RT for 10 min. and then a solution of the BASA of Example 2 (50 mg) in DMF (0.1 ml) is added. The reaction mixture is stirred for 4 h at rt and the solvent is evaporated off. The residue is purified by hplc (reverse-phase C18 column) to give XXXIII (40 mg).

### Example 5

### Synthesis of PEGylated BASA (FIG. 3A)

This synthesis is performed in the same way as described in Example 4, except using the BASA of Example 1 to give XXXII (50 mg).

### Example 6

### Synthesis of PEGylated BACA (FIG. 3C)

The BACA of Example 3 is first treated with ethylenediamine in presence of NaBH₃CN to give ethylenedimine adduct of the BACA, which is then treated with PEG exactly in the same way as described in Example 4 to give XXXVI.

### Example 7

### Assay for E-Selectin Antagonist Activity

Wells of a microtiter plate (plate 1) are coated with E-selectin/hlg chimera (GlycoTech Corp., Rockville, Md.) by incubation for 2 hr at 37° C. After washing the plate 5 times with 50 mM TrisHCl, 150 mM NaCl, 2 mM CaCl₂, pH 7.4 (Tris-Ca), 100 μl of 1% BSA in Tris-Ca/Stabilcoat (SurModics, Eden Prairie, Minn.) (1:1, v/v) are added to each well to block non-specific binding. Test compounds are serially diluted in a second low-binding, round bottomed plate (plate 2) in Tris-Ca (60 μl/well). Preformed conjugates of SLea-PAA-biotin (GlycoTech Corp., Rockville, Md.) mixed with Streptavidin-HRP (Sigma, St. Louis, Mo.) are added to each well of plate 2 (60 μl/well of 1 μg/ml). Plate 1 is washed several times with Tris-Ca and 100 μl/well are transferred from plate 2 to plate 1. After incubation at room temperature for exactly 2 hours the plate is washed and 100 μl/well of TMB reagent (KPL labs, Gaithersburg, Md.) is added to each well. After incubation for 3 minutes at room temperature, the reaction is stopped by adding 100 μl/well of 1 M H₃PO₄ and the absorbance of light at 450 nm is determined by a microtiter plate reader.

### Example 8

### Assay for P-Selectin Antagonist Activity

The neoglycoprotein, sialylLe^{a}-HSA (Isosep AB, Sweden) is coated onto wells of a microtiter plate (plate 1) and the wells are then blocked by the addition of 2% bovine serum albumin (BSA) diluted in Dulbecco's phosphate-buffered saline (DPBS). In a second microtiter plate (plate 2), test antagonists are serially diluted in 1% BSA in DPBS. After blocking, plate 1 is washed and the contents of plate 2 are transferred to plate 1. P-selectin/hlg recombinant chimeric protein (GlycoTech Corp., Rockville, Md.) is further added to each well in plate 1 and the binding process is allowed to incubate for 2 hours at room temperature. Plate 1 is then washed with DPBS and peroxidase-labelled goat anti-human Ig(γ) (KPL Labs, Gaithersburg, Md.) at 1 μg/ml is added to each well. After incubation at room temperature for 1 hour, the plate is washed with DBPS and then TMB substrate (KPL Labs) is added to each well. After incubation at room temperature for 1 hour, the plate is washed with DPBS and then TMB substrate (KPL Labs) is added to each well. After 5 minutes, the reaction is stopped by the addition of 1 M H₃PO₄. Absorbance of light at 450 nm is then determined using a microtiter plate reader.

### Example 9

### Assay for L-Selectin Antagonist Activity

The neoglycoprotein, sialylLe^{a}-HSA (Isosep AB, Sweden) is coated onto wells of a microtiter plate (plate 1) and the wells are then blocked by the addition of 2% bovine serum albumin (BSA) diluted in Dulbecco's phosphate-buffered saline (DPBS). In a second microtiter plate (plate 2), test antagonists are serial diluted in 1% BSA in DPBS. L-selectin/hlg recombinant chimeric protein (GlycoTech Corp, Rockville, Md.) is then added to each well in plate 2. After blocking, plate 1 is washed and the contents of plate 2 are transferred to plate 1. The binding process is allowed to incubate for 2 hours at room temperature. Plate 1 is then washed with DPBS and peroxodase-labelled goat anti-human Ig(gamma) (KPL Labs, Gaithersburg, Md.) at 1 μg/ml is added to each well. After incubation at room temperature for 1 hour, the plate is washed with DPBS and then TMB substrate (KPL Labs) is added to each well. After 5 minutes, the reaction is stopped by the addition of 1 M H₃PO₄. Absorbance of light at 450 nm is then determined using a microtiter plate reader.

### Example 10

### Assay for Effect of a Compound on Cell Rolling in Established Inflammation

Inflammation is induced in normal Swiss Albino mice by intraperitoneal injection of IL-1β (10 ng). After 4 hours, the established inflammatory response is treated with test compounds by intravenous injection. Vehicle is the negative control containing no test compound and mAbs(3) is the positive control containing a cocktail of antibodies to all three selectins (E, 10E9; L, Mel-14; P, RB40.34). Test compound is administered at 50 mg/kg. Rolling of cells on the endothelium is determined by intravital microscopy of the post-capillary venules of the mouse mesentery. Effects of treatment with vehicle, monoclonal antibodies and test compound on cell rolling is monitored for 30 minutes immediately after administration.

All of the above U.S. patents, U.S. patent application publications, U.S. patent applications, foreign patents, foreign patent applications and non-patent publications referred to in this specification are incorporated herein by reference, in their entirety.

From the foregoing it will be appreciated that, although specific embodiments of the invention have been described herein for purposes of illustration, various modifications may be made without deviating from the spirit and scope of the invention.

1. A compound or physiologically acceptable salt thereof, having the formula: 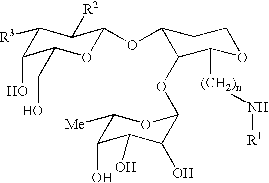
wherein:
n=0-20 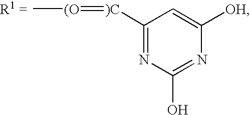
a benzyl amino sulfonic acid, a benzyl amino carboxylic acid, or a second compound or salt thereof having the above formula to form a dimer; 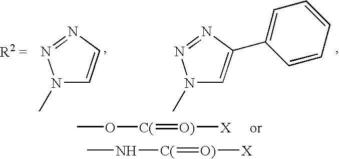
where X is 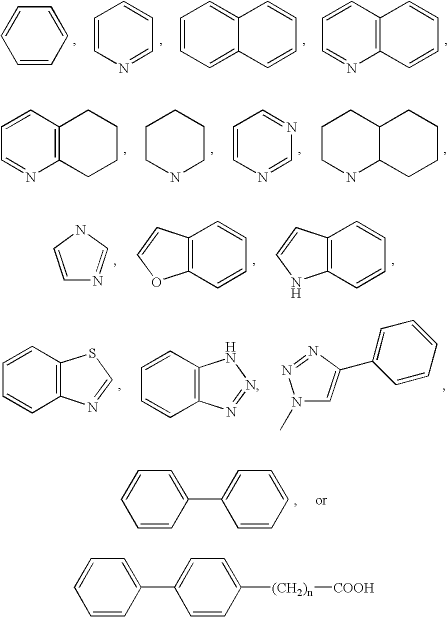
where n=0-10, and any of the above ring compounds may be substituted with one to three of Cl, F, C₁-C₈ alkanyl or OY where Y is H or C₁-C₈ alkanyl;
R³=OH, 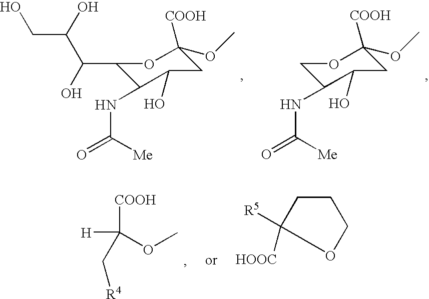
where R⁴ is cyclohexane, t-butane, adamantane, benzene, triazole, or triazole substituted with one to three of Cl, F, C₁-C₈ alkanyl or OY where Y is H or C₁-C₈ alkanyl, and where R⁵ is 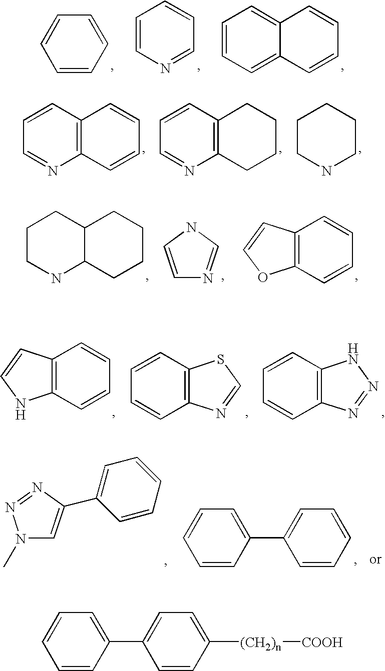
where n=0-10, and any one of the above ring compounds may be substituted with one to three of Cl, F, C₁-C₈ alkanyl or OY where Y is H or C₁-C₈ alkanyl; and
with the proviso that where R¹ is a benzyl amino sulfonic acid and R² or X of R² is aromatic, then R⁴ of R³ is not cyclohexane.

2. The compound or salt thereof according to claim 1 wherein R¹ is a benzyl amino sulfonic acid.

3. The compound or salt thereof according to claim 1 wherein R¹ is a benzyl amino carboxylic acid.

4. The compound or salt thereof according to claim 1 wherein R³ is

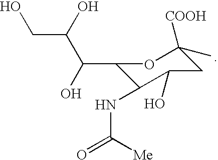

5. The compound or salt thereof according to claim 1 wherein R³ is 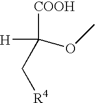
where R⁴ is defined according to claim 1.

6. The compound or salt thereof according to claim 5, where R⁴ is cyclohexane or benzene.

7. The compound or salt thereof according to claim 1 wherein R¹ is

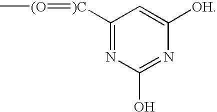

8. The compound or salt thereof according to claim 1 wherein R¹ is a second compound or salt thereof having the formula defined according to claim 1 to form a dimer.

9. The compound or salt thereof according to claim 1 wherein X of R² is

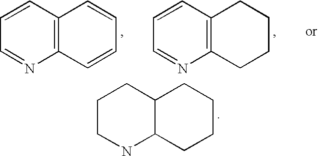

10. The compound or salt thereof according to claim 1 wherein R³ is OH.

11. The compound or salt thereof according to claim 1 wherein R³ is

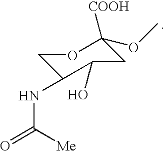

12. The compound or salt thereof according to claim 1 wherein X of R² is

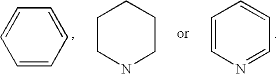

13. The compound or salt thereof according to claim 1 wherein X of R² is

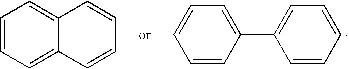

14. The compound or salt thereof according to claim 1 wherein R¹ is linked to the compound or salt thereof by a polyethylene glycol.

15. A composition comprising a compound or salt thereof according to claim 1 in combination with a pharmaceutically acceptable carrier or diluent.

16. A compound or physiologically acceptable salt thereof comprising a compound or salt thereof according to claim 1 further comprising a diagnostic or therapeutic agent.

17. A composition comprising a compound or salt thereof according to claim 16 in combination with a pharmaceutically acceptable carrier or diluent.

18. A method for modulating a selectin-mediated function, comprising contacting a cell expressing a selectin with a compound or salt thereof according to claim 1 in an amount effective to modulate the selectin's function or with a composition according to claim 15 in an amount effective to modulate the selectin's function.

19. (canceled)

20. A method of treating a patient, comprising administering to the patient who is in need of having inhibited the development of a condition associated with an excessive selectin-mediated function, a compound or salt thereof according to claim 1 in an amount effective to inhibit the development of such a condition or a composition according to claim 15 in an amount effective to inhibit the development of such a condition.

21. (canceled)

22. A method of inhibiting rejection of transplanted tissue, comprising administering to a patient who is the recipient of a transplanted tissue, a compound or salt thereof according to claim 1 in an amount effective to inhibit rejection of the transplanted tissue or a composition according to claim 15 in an amount effective to inhibit rejection of the transplanted tissue.

23. (canceled)

24. A method of targeting an agent to a selectin-expressing cell, comprising contacting a cell expressing a selectin with a compound or salt thereof according to claim 16 in an amount effective to target a diagnostic or therapeutic agent to the cell or with a composition according to claim 17 in an amount effective to target a diagnostic or therapeutic agent to the cell.

25.-33. (canceled)
